(19) United States
(12) Patent Application Publication
(10) Pub. No.: US 2021/0212889 A1
(43) Pub. Date: Jul. 15, 2021

(54) CARDIOPULMONARY RESUSCITATION (CPR) QUALITY ADVISOR

(71) Applicants: University of Central Oklahoma, Edmond, OK (US); Wisconsin Alumni Foundation, Madison, WI (US)

(72) Inventors: Nesreen ALSBOU, Edmond, OK (US); Awni AL-SUBU, Cross Plains, WI (US)

(73) Assignees: University of Central Oklahoma, Edmond, OK (US); Wisconsin Alumni Research Foundation, Madison, WI (US)

(21) Appl. No.: 17/149,099

(22) Filed: Jan. 14, 2021

Related U.S. Application Data
(60) Provisional application No. 62/960,885, filed on Jan. 14, 2020.

(51) Int. Cl. A61H 31/00; G16H 50/20

(52) U.S. Cl. CPC A61H 31/005; A61H 31/007; G16H 50/20; A61H 2201/5092; A61H 2230/06; A61H 2230/207; A61H 2230/40; A61H 2201/5046

(57) ABSTRACT

An assistive device for guiding performance of cardiopulmonary resuscitation (CPR) during cardiac arrest (CA), comprising an intelligent device and algorithm that present care givers realtime guidance and feedback on CPR quality using input from multiple invasive and noninvasive biometric monitoring devices. Input is combined and processed using artificial intelligence (AI) techniques to provide performance guidance displayed on a single monitor. Inputs include at least (a) heart rate, (b) end-tidal carbon dioxide—ETCO₂, and (c) regional cerebral oxygen saturation—RSO₂, which are processed to evaluate effectiveness of ongoing CPR and provide performance indicators in real time directed to increasing CPR effectiveness. Artificial intelligence functions evaluate effectiveness of CPR against standards of care as CPR is performed and provides actionable guidance to improve performance. Outputs are produced that include at least (a) performance parameters for compressions, (b) ventilation effectiveness, and (c) indication if return of spontaneous circulation (ROSC) has occurred.

### FIELD OF THE INVENTION

The present invention relates to a system for improving the quality of cardiopulmonary resuscitation (CPR). More particularly, the present invention relates to an intelligent system to provide emergency responders with personalized guidance in preforming cardiopulmonary resuscitation (CPR).

### BACKGROUND OF THE INVENTION

In the United States (US), more than 500,000 children and adults experience a cardiac arrest (CA), and less than 15% survive. The “2010 American Heart Association Guidelines for Cardiopulmonary Resuscitation and Emergency Cardiovascular Care” increased the focus on methods to ensure that high-quality cardiopulmonary resuscitation (CPR) is performed in all resuscitation attempts. There are 5 critical components of high-quality CPR: minimize interruptions in chest compressions, provide compressions of adequate rate and depth, avoid leaning between compressions, and avoid excessive ventilation. Although it is clear that high-quality CPR is the primary component in influencing survival from CA, there is considerable variation in monitoring, implementation, and quality improvement. As such, CPR quality varies widely between systems and locations. Victims often do not receive high-quality CPR because of provider ambiguity in prioritization of resuscitative efforts during an arrest. This ambiguity also impedes the development of optimal systems of care to increase survival from CA. This consensus statement addresses the following key areas of CPR quality for the trained rescuer: metrics of CPR performance; monitoring, feedback, and integration of the patient's response to CPR; team-level logistics to ensure performance of high-quality CPR; and continuous quality improvement on provider, team, and systems levels. Clear definitions of metrics and methods to consistently deliver and improve the quality of CPR will narrow the gap between resuscitation science and the victims, both in and out of the hospital, and lay the foundation for further improvements in the future.

The number of hospital intensive care units (ICU) in the US exceeds 6000. In addition, there are over 327 Pediatric Intensive Care Units (PICU) and 927 Neonatal Intensive Care Units (NICU) in the US caring for 1.4 million infants every year. While the focus in developing the present invention is on reducing infant mortality from cardiac arrest in NICUs and PICUs, the present invention may be used in any ICU.

Approximately 16,000 pediatric patients suffer CA each year in the US. Younger patients, specifically younger than 1 year of age, comprise the majority of pediatric patients with CA, and males are affected in a slightly higher proportion (62%). A collective review of pediatric cases of CA published in 1999 reported a survival rate to discharge of 13%, with good neurologic outcome in 62% of these patients. This early study in pediatric CA identified that patients who sustain a CA in the hospital setting (in-hospital CA) have better survival as compared with patients with CA out of the hospital (out-of-hospital CA) (24% vs. 8.4%, respectively).

Two presently available products on the market and directed to CPR are the ZOLL M Series™ Defibrillator and the LifePak 15. Both of these products only work with Masimo accessories and software. These systems have two operating modes, manual and advisory external defibrillation. When in advisory mode the system will administer shock to the patient automatically and according to the PALS protocol. When in manual mode these systems will let the physician or nurse know when a shock is needed, but will not administer without the physicians or nurses given input. Each of these systems is a portable standalone device that will have patient vitals directly transmitted to the system through dedicated Masimo sensors, cables and attachments. These systems replace existing patient monitoring systems installed in an ICU and provide no interface for connecting to such monitoring system nor using inputs provided by them. In contrast, the present invention integrates the signal outputs of existing patient monitoring systems installed in an ICU. The present invention presents a combined output on a single display, integrating input received from an existing installed base of monitoring systems. Unlike the ZOLL M Series™ Defibrillator and the LifePak 15, the presentment invention implements AI enabled processes that provide actionable feedback to the user on the effectiveness of CPR and indications of the change in actions needed to make CPR being delivered fully effective. The present invention delivers the feedback in both visual and auditory form. Replacement of existing installed systems is not required. This lowers both acquisition cost and training costs.

The outcome during cardiac arrest is highly dependent on immediate and effective cardiopulmonary resuscitation (CPR). The American Heart Association (AHA) Pediatric Advanced Life Support (PALS) guidelines provide guidance on: (1) Quality of CPR; (2) return of spontaneous circulation (ROSC); and (3) Suggestions on next interventions. The present standard of care in ICUs is dependent on numerous monitoring systems requiring that the physician must be accompanied by a team of nurses and technicians in order to view all monitors at the same time, communicate to one another as to parametric status, and then conclude a resuscitation method to intervene on the child. This opens the opportunity for human error, including incorrect reading, a misinterpretation, providing too much or not enough pressure during CPR, or even not providing CPR in a timely manner. Presently, there is no way to combine information from multiple monitors and integrate that with AHA PALS guidelines to provide real time feedback on the quality of CPR. Therefore, the need for accurate and real-time device to monitoring quality of CPR is, therefore, paramount to the improvement of resuscitation outcomes.

### SUMMARY OF THE INVENTION

Pediatric patients experiencing a situation of cardiac arrest must be quickly diagnosed for symptom reversal and execution of Cardiopulmonary Resuscitation (CPR). The American Heart Association (AHA) Pediatric Advanced Life Support (PALS) protocol prescribes executing CPR with two minute intervals and pulse checks. There is a problem that many patients admitted to a Pediatric/Neonatal Intensive Care Unit (PICU/NICU) during cardiac arrest do not receive the necessary care due to ineffective response time or incorrect diagnosis from interpreting data displayed on monitors. To address these problems, the present invention imports data as inputs from at least three types of monitoring devices typically available in a PICU/NICU: Volumetric Capnography monitor, Near Infrared Spectroscopy monitor, and Arterial Line sensing monitor. The present invention provides interfaces for these types of monitors and implements PALS guidance to analyze and communicate to the responder the vitals (e.g., heart rate, respiratory rate and systolic blood pressure [BP]) which exceed their acceptable parameter and enable quick optimization of CPR as needed. In comparison to an Automated External Defibrillator (AED) machine and manual use of PALS protocol alone, the present invention communicates with the person providing CPR to inform what values are falling too low or too high and deliver instructions such as to push harder on the chest or to deliver more oxygen to the patient AED machines only go partially through the steps chronologically without waiting for the bystander or caregiver. PALS is a manual process and must be memorized in order to execute.

The present invention analyzes parameters for Heart Rate, Respiratory Rate, EtCO₂ and rSO₂ (systolic BP may be included) in accordance with AHA PALS protocols. The latter four parameters are noninvasive and are supported by conducted studies utilizing noninvasive readings to replace those of invasive means they reflect as accurately. A noninvasive sensor can be applied on the patient's skin between 15 to 20 seconds and extract a reading within the first 50 seconds. In emergency conditions such as cardiac arrest, this will immensely aid in optimizing CPR by allowing quick readings and conclusions at specific areas of the body to analyze ventilation and compressions. Specific impacts for a patient in cardiac arrest include:
Provide accurate monitoring of oxygen and circulation
Provide non-invasive monitoring of oxygen and circulation
Provide continuous monitoring of oxygen and circulation
Reduce responder traffic per patient
Organize information
Real-time response to CA

It is an objective of the present invention to provide an intelligent device and algorithm to read, process, and output data with as close to real time sampling, processing, and communication as to not disrupt the resuscitation process.

It is an object of the present invention to provide an intelligent device and algorithm that presents care givers realtime guidance and feedback on CPR quality using input from multiple monitors, invasive or noninvasive. The present invention combines input from multiple monitoring devices and uses artificial intelligence (AI) techniques, including machine learning, to provide guidance displayed on a single monitor during CPR.

It is an object of the present invention to import data as inputs from at least three types of monitoring devices typically available in a PICU/NICU: Volumetric Capnography monitor, Near Infrared Spectroscopy monitor, and Arterial Line sensing monitor.

In one aspect, the present invention provides an electronic interface for receiving signal input from any of multiple invasive and noninvasive biometric monitoring devices that measure patient vital signs comprising at least heart rate, end-tidal carbon dioxide—ETCO₂, and regional cerebral oxygen saturation—RSO₂.

In one aspect, the present invention receives inputs to an AI enabled processing device including at least (1) heart rate, (2) end-tidal carbon dioxide—ETCO₂, and (3) regional cerebral oxygen saturation—RSO₂, which together are processed by the device to evaluate the effectiveness of ongoing CPR and provide performance indicators in real time directed to increasing CPR effectiveness for a patient experiencing CA.

In another aspect, the present invention includes an artificial intelligence function, that evaluates effectiveness of CPR against standards of care as CPR is performed and provides actionable guidance directed to how to make the CPR being delivered fully PALS compliant.

In another aspect, the present invention produces outputs as corrective action indicators that include at least (1) performance parameters for compressions (e.g., press harder, softer, or keep doing what you're doing), (2) ventilation effectiveness, and (3) indication if return of spontaneous circulation (ROSC) has occurred.

In another aspect, the present invention provides a Graphical User Interface (GUI) that delivers in substantially realtime actionable visual and aural guidance and feedback including at least one of limiting or actuating changes and rate of changes of at least chest compression to affect alteration in patient vital signs, the guidance including corrective indicators directed to CPR performance adjustments to achieve therapeutic motion technique responsive to deviation from said personalized boundary values.

In one aspect, the present invention provides a method for guiding delivery and evaluating performance of cardiopulmonary resuscitation (CPR) during cardiac arrest (CA), the method including receiving signal input from any of multiple invasive and noninvasive biometric monitoring devices that measure patient vital signs, evaluating received patient vital sign data against personalized boundary parameter values compliant with American Heart Association (AHA) Pediatric Advanced Life Support (PALS) protocols.

In another aspect, the present invention provides a Graphical User Interface (GUI) that delivers in substantially realtime actionable visual and aural guidance and feedback including at least one of limiting or actuating changes and rate of changes of at least chest compression to affect alteration in patient vital signs, said guidance including corrective indicators directed to CPR performance adjustments to achieve therapeutic motion technique responsive to deviation from said personalized boundary values.

In another aspect, the present invention may be configured for use in either a relatively fixed institutional setting including at least an emergency room (ER) and an ICU, or a dynamic environment including at least emergency vehicles and personal mobile devices.

### BRIEF DESCRIPTION OF THE DRAWINGS

FIG. 1 is a non-limiting block diagram showing typically available patient monitors providing specific input data to the AI enabled device of the present invention and the output resulting from evaluation of the data by the AI enabled device.

FIG. 2 is a non-limiting diagram showing a schematic of the present invention in prototype form.

FIG. 3 is a non-limiting diagram showing VGA Pinout for a INVOS 5100C display.

FIG. 4 is a non-limiting diagram showing flow and pseudocode that indicates if the heart rhythm of a patient is shockable or requires CPR.

FIG. 5 is a non-limiting diagram showing a Simulink testing structure for an ECG input.

FIG. 6 is a non-limiting diagram showing filtered ECG signals produced using the Simlink testing structure shown in FIG. 5.

FIG. 7 is a non-limiting diagram showing the general analysis of exemplary inputs to the present invention which for purposes of the experimental design is referred to as “Smart Medical System.”

FIG. 8 is a non-limiting diagram showing general analysis of EtCO₂ inputs to the present invention which for purposes of the experimental design is referred to as “Smart Medical System.”

FIG. 9 is a non-limiting diagram showing general analysis of rSO₂ inputs to the present invention which for purposes of the experimental design is referred to as “Smart Medical System.”

FIG. 10 is a non-limiting diagram showing general analysis of Respiratory Rate inputs to the present invention which for purposes of the experimental design is referred to as “Smart Medical System.”

FIG. 11 is a non-limiting diagram showing general analysis of Heart Rate inputs to the present invention which for purposes of the experimental design is referred to as “Smart Medical System.”

FIG. 12 is a non-limiting diagram showing data flow through source code implemented as MATLAB code executed from SIMULINK for prototype development using the “ECGSignalProcessing” built-in tool of SIMULINK to filter raw ECG signals into the distinct amplitudes of concerns that would appear on capnography of a monitor.

FIG. 13 is a non-limiting diagram showing an electrical circuit of the present invention designed to physically connect medical monitors typically available in a NICU/PICU to the “Smart Medical System” of the present invention.

FIG. 14 is a non-limiting diagram showing a prototype GUI developed to present five preferred specifications

FIG. 15 is a non-limiting diagram showing the prototype GUI of the present invention where two additional preferred specifications are implemented.

FIG. 16 is a non-limiting diagram showing a prototype design of the present invention.

### DETAILED DESCRIPTION

In brief:

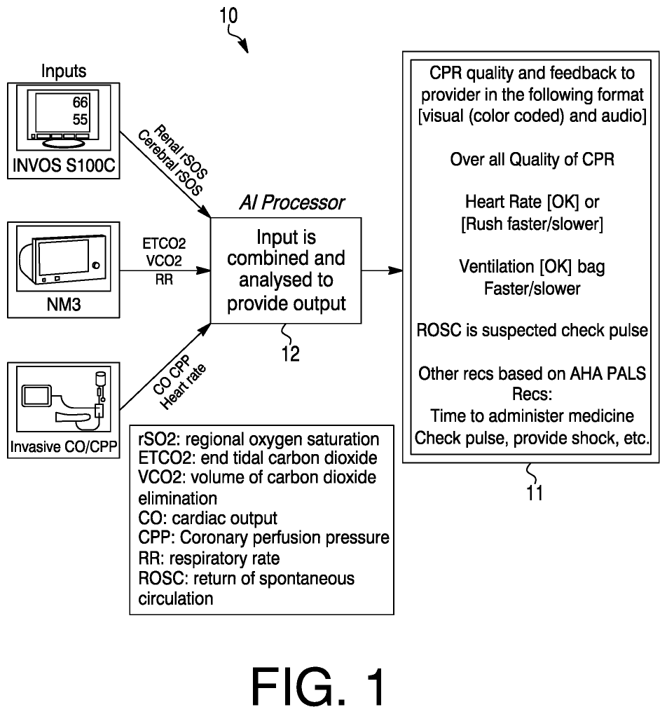

Referring to FIG. 1, the present invention receives inputs to an AI enabled processing device including at least (1) heart rate, (2) end-tidal carbon dioxide—ETCO2, and (3) regional cerebral oxygen saturation—RSO2, which together are processed by the device to evaluate the effectiveness of ongoing CPR and provide performance indicators in real time directed to increasing CPR effectiveness for a patient experiencing CA. The present invention includes an artificial intelligence function, that evaluates effectiveness of CPR against standards of care (e.g., AHA PALS guidelines) as CPR is performed and provides actionable guidance directed to how to make the CPR being delivered fully effective. The present invention may be configured for use in either a relatively fixed institutional setting including at least an emergency room (ER) and an ICU, or a dynamic environment including at least emergency vehicles and personal mobile devices.

The present invention produces outputs that include at least (1) performance parameters for compressions (e.g., press harder, softer, or keep doing what you're doing), (2) ventilation effectiveness, and (3) indication if return of spontaneous circulation (ROSC) has occurred. The guidance provided by these performance parameters is presented on a single monitor, along with an indication of ventilation effectiveness and an ROSC event. Guidance may be presented in either or both visual and aural form.

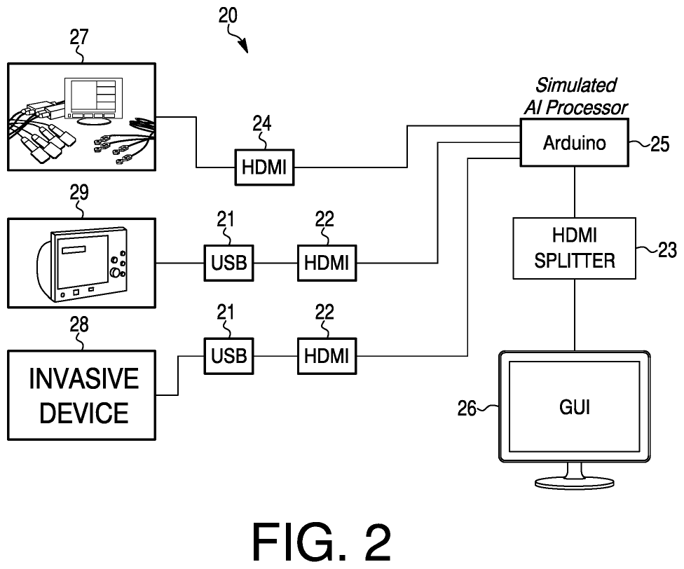

Referring to FIG. 2, at least three monitoring devices will deliver data to the circuit board of an AI enabled processor of the present invention. Data delivery will be via USB or HDMI specifications or analogs thereof. In the AI enabled processor (shown simulated in an Arduino) processor code triggers which information is necessary to be shown on the single display monitor and presents the required information in a user designated or a default format.

The present invention provides an intelligent device and algorithm that presents care givers realtime guidance and feedback on CPR quality using input from multiple invasive and noninvasive monitoring devices. The present invention combines input from these multiple monitoring devices and uses artificial intelligence (AI) techniques to integrate that input with AHA PALS guidelines to provide real time guidance on the quality of ongoing CPR, which is displayed on a single monitor during CPR and may include actionable aural guidance.

The present invention provides a Graphical User Interface (GUI) that delivers in substantially realtime actionable visual and aural guidance and feedback including at least one of limiting or actuating changes and rate of changes of at least chest compression to affect alteration in patient vital signs, the guidance including corrective indicators directed to CPR performance adjustments to achieve therapeutic motion technique responsive to deviation from said personalized boundary values.

The present invention provides a method for guiding delivery and evaluating performance of cardiopulmonary resuscitation (CPR) during cardiac arrest (CA), the method including receiving signal input from any of multiple invasive and noninvasive biometric monitoring devices that measure patient vital signs, evaluating received patient vital sign data against personalized boundary parameter values compliant with American Heart Association (AHA) Pediatric Advanced Life Support (PALS) protocols, providing a Graphical User Interface (GUI) that delivers in substantially realtime actionable visual and aural guidance and feedback including at least one of limiting or actuating changes and rate of changes of at least chest compression to affect alteration in patient vital signs, the guidance including corrective indicators directed to CPR performance adjustments compressions (e.g., press harder, softer, or keep doing what you're doing) to achieve therapeutic motion technique responsive to deviation from personalized boundary values. Indicators of delivered CPR quality are measured by at least ventilation effectiveness, and return of spontaneous circulation (ROSC).

In detail:

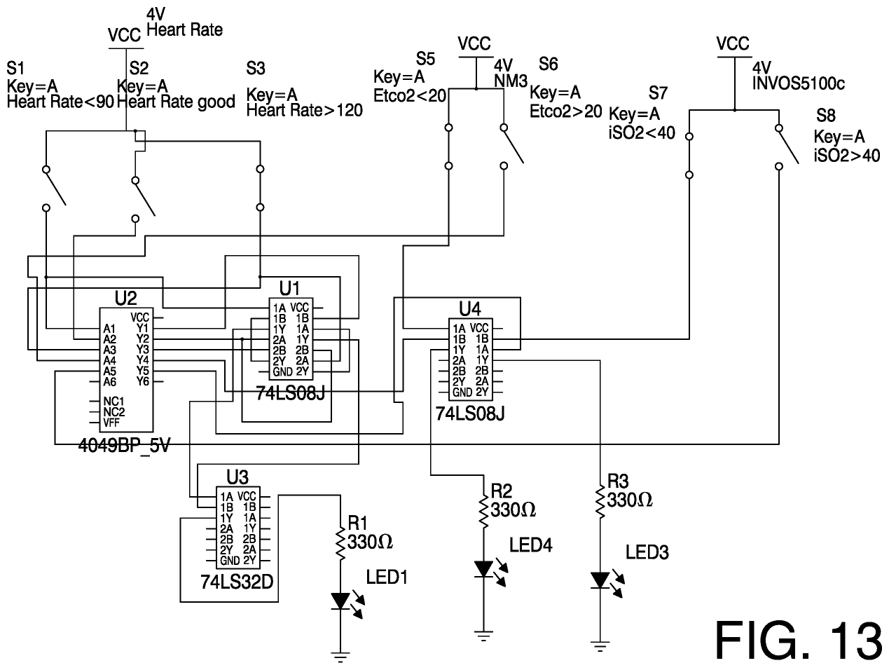

Referring now to FIG. 1, the present invention 10 comprises both hardware and AI enabled software. The hardware includes a single display 11, an AI enabled processing device 12, including a power source (e.g., battery or power supply). The display 11 may comprise a liquid-crystal-display, light-emitting-diode display, or any other type of display suitable for presenting real-time information. The display screen should be compatible with a control board to enable interface between external systems and the display monitor. A 12 volt, more or less, power source may be used to power the system. An Arduino Uno (or any type of Arduino was used to develop a prototype of the present invention. A specific purpose processor 12 will be produced to carry out the needs of this system comprising the present invention 10. A prototype processor circuit board is shown in FIG. 13, however a purpose built circuit board will be required.

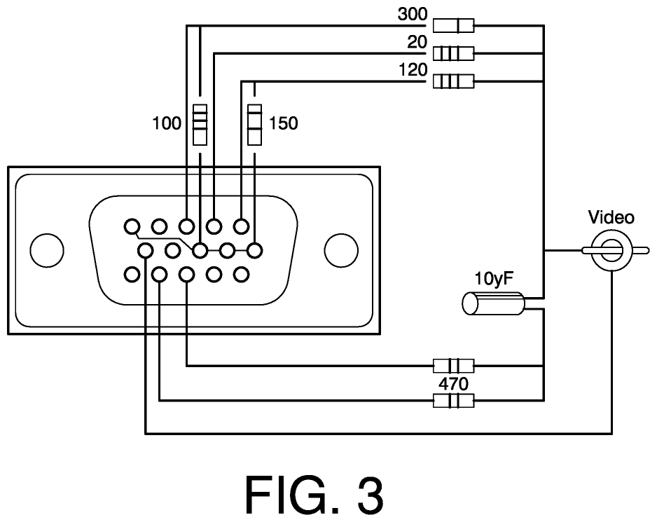

Now referring to FIG. 2, USB cables 21, USB to HDMI adapters 22, HDMI splitters 23, and HDMI 24 cables may all be required to connect the AI enabled processor 25 of the present invention 20 to transfer data and display actionable guidance on the single display monitor 26. The AI enabled software of the present invention incorporates machine learning (a subset of AI technology) to provide a code set that may receive, analyze, process, and output data received by non-invasive devices (27, 29) and invasive devices 28 monitoring ventilation and oxygenation. The data will come first from at least three monitoring devices (27, 28, 29) by direct wire connection. For example, an INVOS 5100C 27 may transmit data directly from its HDMI output via an HDMI CABLE 24 to the circuit board of the AI enabled processor 25. A Respironics NM3 29 and an invasive device 28 may output data via USB. The USB 21 connection transfers a signal into a USB-to-HDMI converter. 22 The HDMI converter 22 transmits the signal data into the circuit board of the AI enabled processor 25 for evaluating the data based on AHA PALS and other parameters derived from clinical research. Once this information has processed, the performance guidance necessary to optimize CPR will output to the HDMI splitter 23. The code determines which HDMI port to display by sending a high interrupt output to pin 13 which is the triggering pin of the HDMI to display 26. The other HDMI displays will continue to run in the background as embedded screens of the GUI. When their performance parameter becomes necessary or dangerous, the signal applied to HDMI pin 13 (shown in FIG. 3) will trigger the display 25 and the display 25 will change in response.

Exemplary, nonlimiting steps to using the hardware and software are as follows when configuring the present invention 20 to operate with an INVOS 5100C 27, Respironics NM3 29 and a typical Invasive Device 29:
The INVOS 5100C 27 transmits data directly via an HDMI cable 24 to the circuit board of the AI enabled processor 25.
Both the Respironics NM3 29 and the Invasive Device 28 transmit data via a USB cable 21 directly from the devices to a USB-to-HDMI adapter/converter 22. The adapter/converter 22 is connected directly to the purpose built circuit board in the AI processor 25 of the present invention 20.
The data going into the circuit board of the AI Processor 25 will be analyzed and processed to determine which screen should be shown because associated performance is a concern based on AHA PALS and clinical research findings.
The AI enabled processor (shown in FIG. 2 as an Arduino) may be coded to trigger the needed pin from the HDMI splitter 23 to display the necessary performance guidance on the display monitor 25.
The steps of this process will repeat until the patient is stable. The software may have a default setting coded to display all monitors (27, 28, 29) and a green outline on the edges of the screen.

### Development Results

In a preferred embodiment as shown in FIG. 1, the present invention 10 is designed to take in eight major values declared to be significant during cardiac arrest, the values provided from three external monitors: typically, the INVOS 5100C, Respironics NM3, and the particular invasive device of the hospital which may vary from hospital to hospital. The present invention 10 provides an AI enabled processor 12 and one display monitor 11, where the AI enabled processor 12 (simulated by an Arduino in the prototype) interfaces the other three monitors. Signals come in at real-time and output resuscitation methods that optimize CPR, ventilation, or the necessity to deliver shock. In the prototype, software is configured to include C code to transfer inputs from the three devices directly connected to the patient to the AI enabled processor 12, algorithms to process the data via Simulink, algorithms to automate the input through the AHA PALS protocol, and a graphic user interface (GUI) 11 to display capnography and communication. The GUI displayed on the monitor (11, FIG. 1; 26, FIG. 2) provides the capability to rotate through four display states based on the following:

1) CPR Quality [from INVOS 5100C and Respironics NM3]
a) Determines CPR need
b) Determines Defibrillation need

2) Heart Rate Range [from Invasive Device]
a) Determines compression pressure
b) Determines compression speed

3) Ventilation [NM3]
a) Determines bag speed
b) Determines external oxygen increase or decrease

4) Return of Sudden Circulation (ROSC) [from INVOS 5100C and Respironics NM3]
a) Requiring pulse check

The hardware of the present invention 20, FIG. 2 provides a single interfacing display monitor 26, FIG. 2 to receive inputs either wired or wirelessly and an output for areas of concerns or necessary actions with visual representation (graphs, plots, displaying time) as well as aural communication with the user (e.g., physician). With this prototype design, the present invention solves the problems of disparate displays in current hospital environments, and accounts for drawbacks from current solutions.

Currently, the solutions for patients who need CPR are the AHA PALS protocol and using multiple monitors to display measures of the effectiveness of CPR. There are also invasive solutions as well; however, the risk of bacteria and the time it takes to properly apply an invasive sensor is greatly increased. While extremely accurate, less than 50% of patients receive this type of care. In the AHA PALS protocol, there is no feedback for quality of CPR patients are receiving. The INVOS 5100C and the NM3 can be used to monitor the effectiveness of CPR, but there needs to be a technician to visually sift through the information because of the disorganized and overwhelming amount of data, as well as increased time it takes to come to the best solution. Further, the invasive method may not be suitable for all patients. These all pose as disadvantages in current solutions. To address these particular problems, the present invention 10, FIG. 1 transforms measured data into actionable guidance in substantially real-time and organizes information to be meaningfully displayed. The present invention 20, FIG. 2 takes all the information down to one monitor 26, FIG. 2 and shows values as they are necessary. Enabling use of non-invasive values that can replace invasive values will bring the overall risk down and cut down on time to apply sensors and receive readings, making it possible for more patients to receive proper treatment. The present invention 10, FIG. 1; 20, FIG. 2 provides specific AHA PALS compliant guidance to make CPR implementation more effective, providing patients with a treatment strategy that is best for them. The present invention 10, FIG. 1; 20, FIG. 2 communicates the quality of CPR being performed based on feedback and helps increase the chances of ROSC, as well as predict when ROSC would occur. The present invention 12, FIG. 1; 20, FIG. 2 is designed to evaluate the effectiveness of CPR using the AI enabled processor 10, FIG. 1; 25, FIG. 2 to analyze several different sensor signals, which will save on time, minimize risk, and make this treatment accessible to all patients, thus proving to be the best solution for people suffering from cardiac arrest.

The software and hardware of the present invention 10, FIG. 1; 20, FIG. 2 have a plurality of key aspects. The software has four key parts to achieve an automated system using artificial intelligence:
1) The C code to take inputs from at least three different monitors and pass them into the AI enabled processor;
2) The algorithms to process the data via Simulink or analogs;
3) The algorithms to automate the inputs through the AHA PALS protocol;
4) The graphic user interface (GUI) adapted to display actionable guidance.

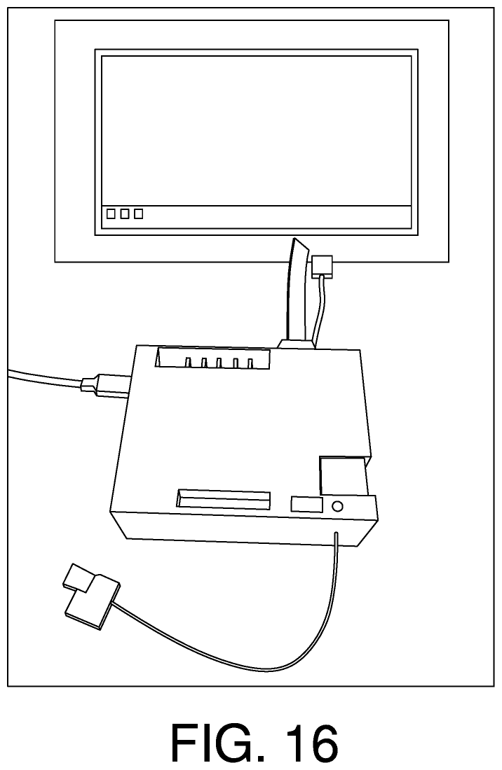

In the PICU/NICU environment, the patient will have numerous non-invasive and invasive sensors reading parametric values. For the first part of the software breakdown, the computer code (C language or analogs) will take in parametric values from at least the three main monitors via direct wire or wireless connection. A typical hospital environment has an INVOS 5100C equipped with a VGA port. This port can be connected to the AI enabled processor and display outputs directly. The VGA pinout is shown in FIG. 3, which enables the connection needed for displaying the capnography for Renal and Cerebral rSO2 (File: VGA Pinout). With this pin diagram, the visual can be adjusted by receiving a low or high voltage to hide or bring up the screens and values needed. This was implemented in the prototype shown in FIG. 16.

The Respironics NM3 (29, FIG. 2) typical of hospital PICU/NICU settings has a USB output and open interface where data is transferred using the USB connection. The particular issue here is that the USB may not transfer the data and the audio at the same time, so an adapter (22, FIG. 2) for DVI or VGA may be required in order to accurately transfer the plots from the Respironics NM3 (29, FIG. 2) to the AI enabled processor (25, FIG. 2) of the present invention. The invasive device (28, FIG. 2) and interface requirements will be site specific and may require an adapter. However, the present prototype design is adapted for a USB capability that provides input into the AI enabled processor (25, FIG. 2). Other connector types may also be used, including at least HDMI. The data values are transferred into the AI enabled processor (25, FIG. 2) and sorted by the computer code before being processed (by the Simulink system in the prototype). The algorithms of the present invention (20, FIG. 2) consist of at least two different parts: first to synchronize the sampled data and second to process the sampled data. To synchronize the data, the Master-Slave approach of Data Acquisition (DAQ) may be applied (Acquire Data). The data will come into the DAQ and be synchronized using the computer code. The values may be stored in an array and execute based on a Master clock. This way, all values output based on the same timing. To process the data in the prototype, the Simulink system filters out the distortion as well as any outliers so that accurate readings could be made. The AHA PALS protocol for patients undergoing cardiac arrest currently procedurally walks physicians through resuscitation in a manual process without feedback as to effectiveness, but the AI enabled processor (25, FIG. 2) of the present invention (20, FIG. 2) provides real-time, actionable guidance in successive loops.

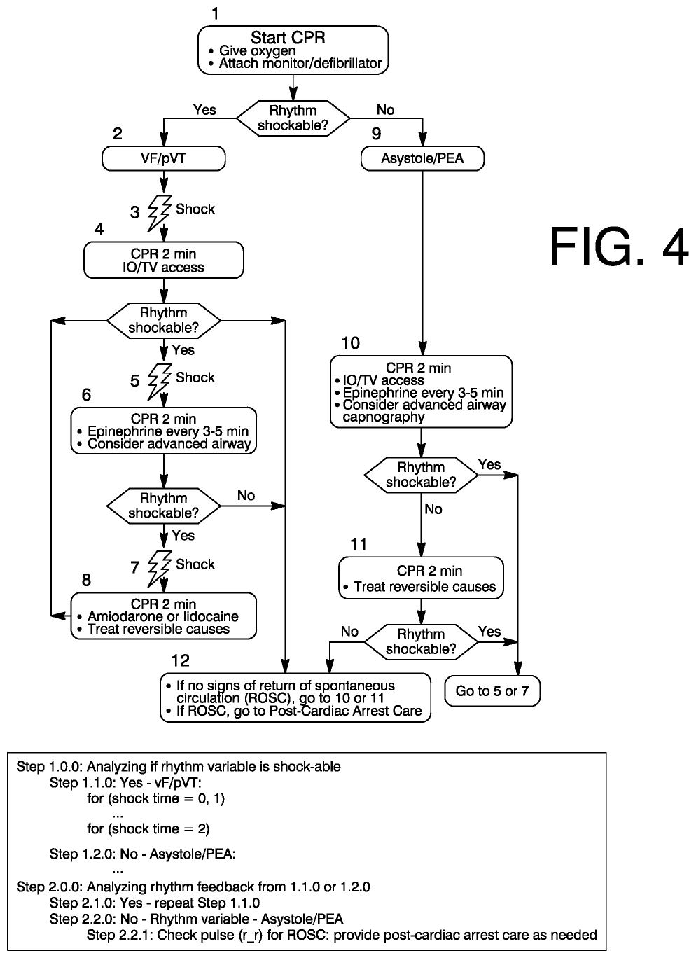

FIG. 4 shows CPR procedural flow and a portion of pseudocode that describes if the heart rhythm is shockable or requires CPR. The steps after this optimize the CPR based on analysis of parametric values feeding into the AI enabled processor (FIG. 2, 25).

The hardware has two key parts to achieve the level of convenience, effectiveness, and reliable communication which will enable the physician to optimize resuscitation:

1) The monitor to display values with direct wire connection; and

2) The visual and audio capabilities for ROSC monitoring and protocol to administer.

One main problem in a typical hospital ICU set-up is the requirement of numerous people in the room to view all of the monitors simultaneously. This can be difficult if there is an issue with the number of physicians, nurses, and technicians on duty. The timing of notifying and gathering everyone in the room to read the monitors can also affect the opportunity to begin resuscitation for the patient. Based on research and deliverable specifications from physicians, the present invention (10, FIG. 1; 20, FIG. 2) provides at least the following or equivalent features and capabilities:

Two USB Ports
Receive output from Respironics NM3 or analogs
Receive output from (site specific) non-Invasive and invasive devices

One VGA Port
Receive output from INVOS 5100C or analogs

One DVI Port
Receive output from INVOS 5100C or analogs as back-up

One Regular Display Port
Receive output from INVOS 5100C or analogs as back-up

One Audio Output
Announce necessary resuscitation instructions to physician
Announce desired feedback by physician specification on the GUI

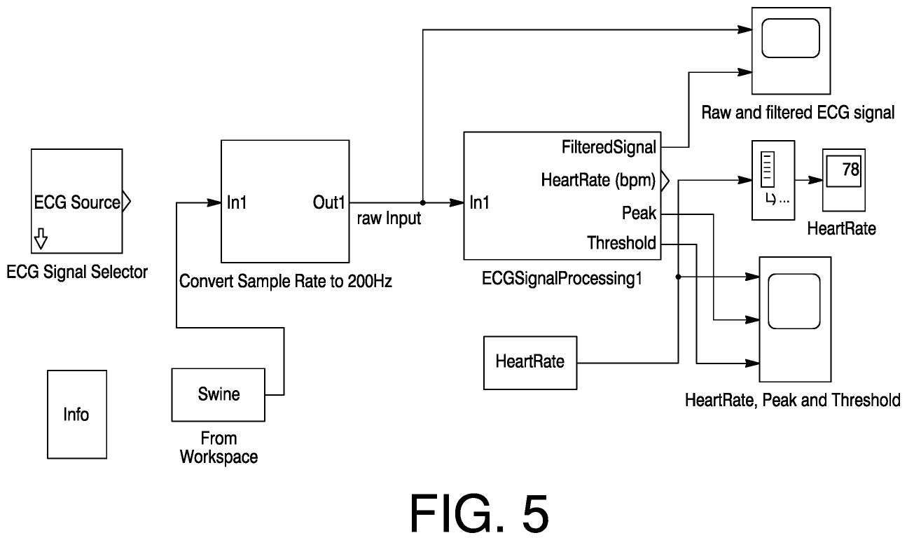

Referring to FIG. 5, in the design phase, Simulink was used to generate a desired input of signals via sample Swine Test data. Experimentation for the prototype used a range of sample data stored in MATLAB and PhysioNet data resources for complex physiological signals. Testing demonstrated that data can be filtered to clarify peaks and magnitudes that are important during ROSC analysis. The sample data included an Electrocardiogram (ECG) signal and was generated via computer C code.

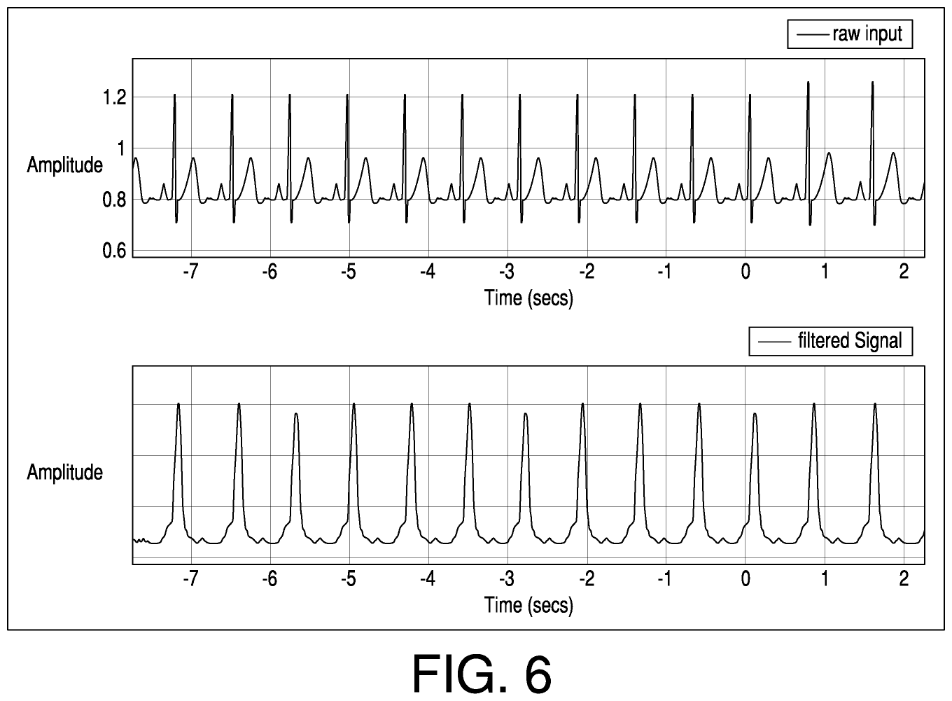

Referring to FIG. 6, the display shows both the original ECG signal and the filtered version which only has the magnitudes. The computer C code calculates the heart rate. The value outputs as binary and is then converted to decimal numbers to be displayed via the built-in decoder. The coding block regenerates the correct heart rates to be displayed. A similar coding block structure performs the analysis of the other signals to be collected. In order to optimize CPR, the focus is on two major procedures: chest compressions and ventilation. In order to optimize chest compressions, it may be necessary to monitor EtCO₂ using the invasive method of measuring the heart rate which is the preexisting method in many PICU environments. From the monitor, the signal may be intercepted using the real-time digital output on a Respironics NM3 or analog. Based on parameters determined from research, the ideal signal of what the heart rate trend is for the patient of a similar category may be graphed to serve as a reference to assure the care giver performing CPR is executing effective chest compressions. A visual representation of the ROSC goal will assist in guiding the care giver with how much pressure to apply per compression and how fast to execute the compressions. In order to optimize ventilation, it is necessary to monitor rSO₂ and use an audible noise such as a chirp to tell when to give the rescue breaths. This performance pattern may change because some patients that are in the hospital may already be on a controlled ventilation system, whereas emergency admitted patients may require more immediate attention.

### Experimental Design

Based on findings from a literature review and code designs, algorithms for AHA PALS were drafted along with a source code. The algorithms were produced as “troubleshooting trees” by going case-by-case of the AHA PALS protocol. A troubleshooting tree was drawn up for the top level system of the present invention and for each exemplary input from a monitor. The troubleshooting trees present the flow for evaluating the effectiveness of ongoing CPR and provide performance indicators in real time directed to increasing CPR effectiveness for a patient experiencing CA.

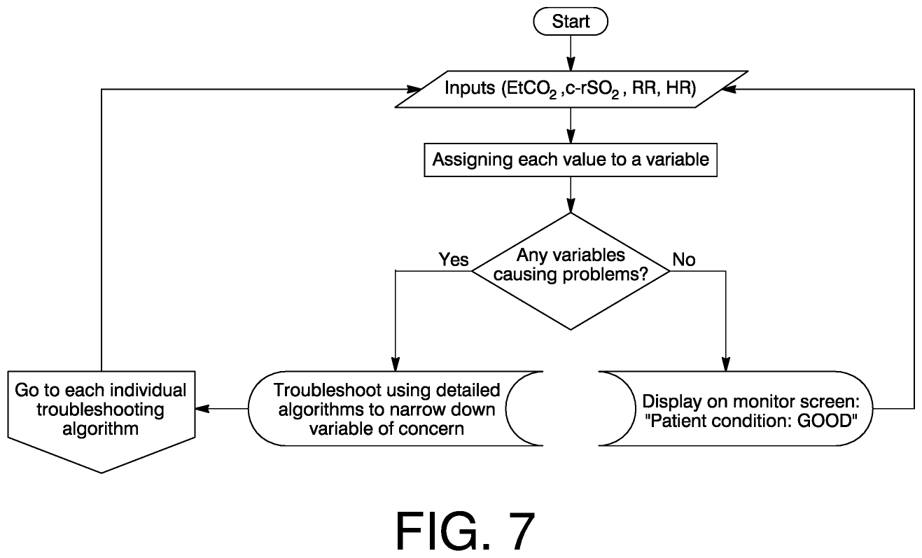

The top level system of the present invention is represented in FIG. 7. The troubleshooting tree shown in FIG. 7 depicts the general analysis of exemplary inputs to the present invention which for purposes of the experimental design is referred to as “Smart Medical System.” As shown in FIG. 7, the present invention receives inputs including at least (1) heart rate, (2) end-tidal carbon dioxide—EtCO₂, and (3) regional cerebral oxygen saturation—RSO₂, which together are processed by the AI enabled processing device to evaluate the effectiveness of ongoing CPR and provide performance indicators in real time directed to increasing CPR effectiveness for a patient experiencing CA.

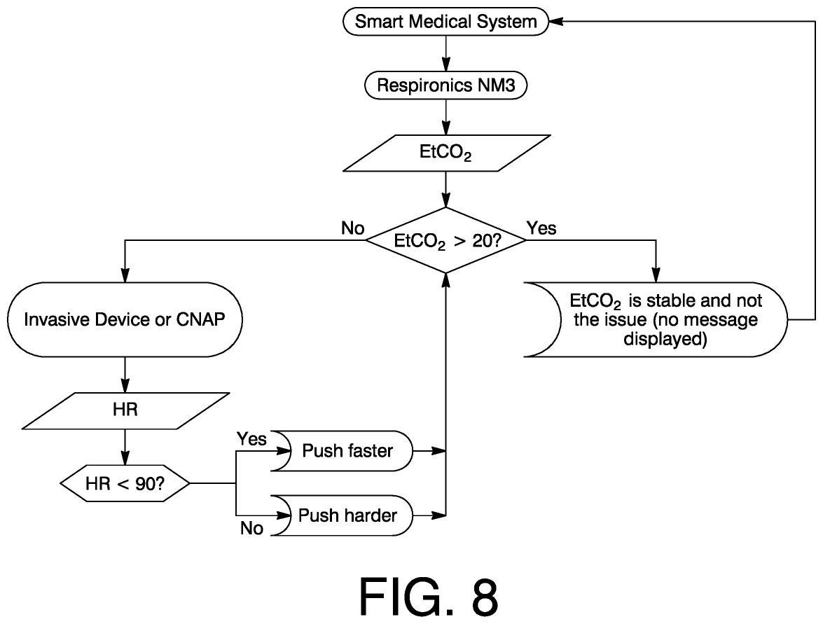

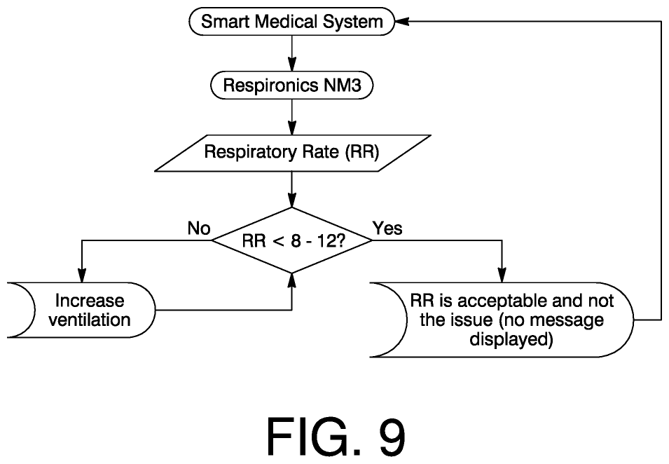

The two cases of EtCO₂ and rSO₂ are shown in FIG. 8 and FIG. 9, respectively. These parameters depended on the Heart Rate primarily for CPR execution. The rSO₂ algorithm allowed the Heart Rate to be from 100-120; however, for the prototype using MATLAB code, it was designed to take 100 as the goal Heart Rate. As MATLAB was determined to be the finalized coding language for the prototype, there were limitations of how many embedded loops could be written before the processor began slowing down immensely. Thus, it was concluded to remove the Heart Rate range of 100-120 and instead replace it with 100. However, this would not necessarily be a limitation if an alternative coding language is used.

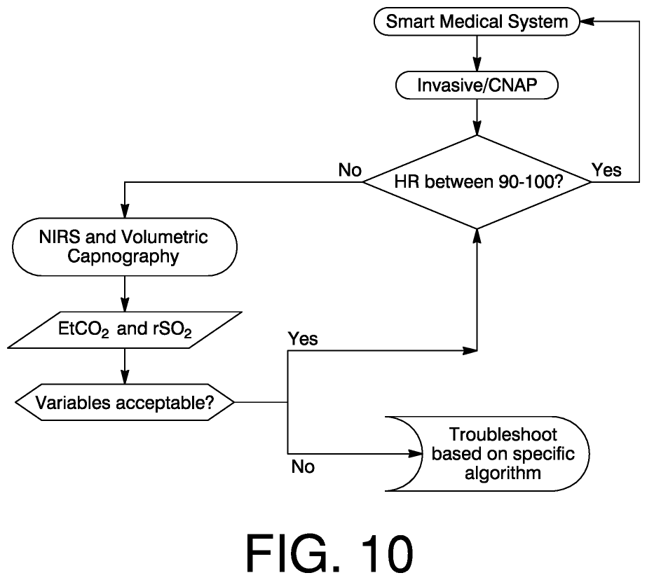

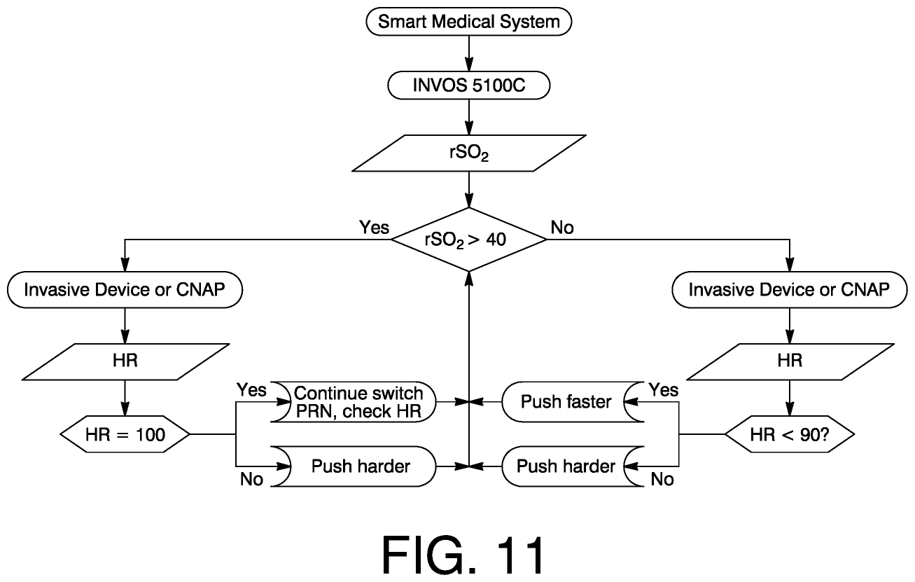

The two cases for Respiratory Rate and Heart Rate are shown in FIG. 10 and FIG. 11, respectively. Respiratory rate is independent and correlates to ventilation alerts only. Heart Rate is an independent reading; however, EtCO₂ and rSO₂ depend on it for determining compression pressure and speed. It was at first considered as requiring age; however, further clarification surprisingly determined that age did not factor into the algorithm as initially considered.

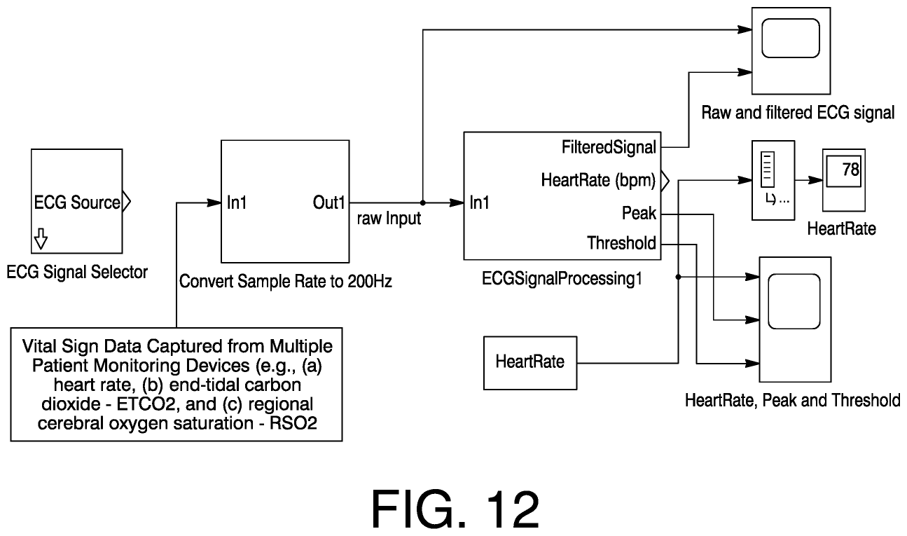

Referring to FIG. 12, the present invention (10, FIG. 1; 20, FIG. 2) provides at least two functions. The first is transmission of the desired signals from the patient monitors, and combines them into the AI enabled processor. The second part is analyzing the signals to give the desired output. In the design phase for the prototype hardware and implementation phase for software, the software utilized is Simulink, a ATLAB-based graphical programming environment for analyzing multi-domain systems, simulation, and modeling. Alternative approaches are possible and anticipated.

The source code implemented for the prototype is a MATLAB code executed from SIMULINK. The prototype system of the present invention (10, FIG. 1; 20, FIG. 2) utilizes the “ECGSignalProcessing” built-in tool of SIMULINK to filter raw ECG signals into the distinct amplitudes of concerns that would appear on capnography of a monitor. This system begins with a variety of patients in the “ECG Signal Selector” area. From there, the user can set which patient they are analyzing. That ECG signal is then transferred into the “Convert Sample Rate to 200 Hz” section. In this area, the raw data is converted to sampling rate 200 Hz to be processed in the further section. The 200 Hz is selected as default in MATLAB where the filter bandwidth choice will be enough. These are default values intended to support further clinical research to provide a range of values that may be optimized for specific applications. The source code implemented for prototype testing is presented in the following listing:

x=0.01:0.01:2;
default=input(‘Press 1 if u want default ecg signal else press 2:\n’);
if(default==1)

```
  li=30/72;
  a_pwav=0.25;
  d_pwav=0.09;
  t_pwav=0.16;
  a_qwav=0.025;
  d_qwav=0.066;
  t_qwav=0.166;
  a_qrswav=1.6;
  d_qrswav=0.11;
  a_swav=0.25;
  d_swav=0.066;
  t_swav=0.09;
  a_twav=0.35;
  d_twav=0.142;
  t_twav=0.2;
  a_uwav=0.035;
  d_uwav=0.0476;
  t_uwav=0.433;
```

else

rate=input(‘\n\nenter the heart beat rate :’);
li=30/rate;
%p wave specifications
fprintf(‘\n\np wave specifications\n’);
d=input(‘Enter 1 for default specification else press 2: \n’);
if(d==1)

a_pwav=0.25;
d_pwav=0.09;
t_pwav=0.16;

```
else
  a_pwav=input(‘amplitude = ’);
  d_pwav=input(‘duration = ’);
  t_pwav=input(‘p-r interval = ’);
  d=0;
end
%q wave specifications
fprintf(‘\n\nq wave specifications\n’);
d=input(‘Enter 1 for default specification else press 2: \n’);
if(d==1)
```

a_qwav=0.025;
d_qwav=0.066;
t_qwav=0.166;

```
else
  a_qwav=input(‘amplitude = ’);
  d_qwav=input(‘duration = ’);
  t_qwav=0.166;
  d=0;
end
%qrs wave specifications
fprintf(‘\n\nqrs wave specifications\n’);
d=input(‘Enter 1 for default specification else press 2: \n’);
if(d==1)
```

a_qrswav=1.6;
d_qrswav=0.11;

```
else
  a_qrswav=input(‘amplitude = ’);
  d_qrswav=input(‘duration = ’);
  d=0;
end
%s wave specifications
fprintf(‘\n\ns wave specifications\n’);
d=input(‘Enter 1 for default specification else press 2: \n’);
if(d==1)
```

a_swav=0.25;
d_swav=0.066;
t_swav=0.09;

```
else
  a_swav=input(‘amplitude = ’);
  d_swav=input(‘duration = ’);
  t_swav=0.09;
  d=0;
end
%t wave specifications
fprintf(‘\n\nt wave specifications\n’);
d=input(‘Enter 1 for default specification else press 2: \n’);
if(d==1)
```

a_twav=0.35;
d_twav=0.142;
t_twav=0.2;

```
else
  a_twav=input(‘amplitude = ’);
  d_twav=input(‘duration = ’);
  t_twav=input(‘s-t interval = ’);
  d=0;
end
%u wave specifications
fprintf(‘\n\nu wave specifications\n’);
d=input(‘Enter 1 for default specification else press 2: \n’);
if(d==1)
```

a_uwav=0.035;
d_uwav=0.0476;
t_uwav=0.433;

```
else
  a_uwav=input(‘amplitude = ’);
  d_uwav=input(‘duration = ’);
  t_uwav=0.433;
  d=0;
end
```

```
end
  pwav=p_wav(x,a_pwav,d_pwav,t_pwav,li);
  %qwav output
  qwav=q_wav(x,a_qwav,d_qwav,t_qwav,li);
  %qrswav output
  qrswav=qrs_wav(x,a_qrswav,d_qrswav,li);
  %swav output
  swav=s_wav(x,a_swav,d_swav,t_swav,li);
  %twav output
  twav=t_wav(x,a_twav,d_twav,t_twav,li);
  %uwav output
  uwav=u_wav(x,a_uwav,d_uwav,t_uwav,li);
  %ecg output
  ecg=pwav+qrswav+twav+swav+qwav+uwav;
  figure(1)
  plot(x,ecg);
```

Referring to FIG. 13, an electrical circuit was designed to physically connect typical medical monitors to the “Smart Medical System” of the present invention (10, FIG. 1; 20, FIG. 2). The electrical circuit consists of digital logic gates taking the display pin of each medical device and running them through the microprocessor. The initial design in the prototype includes switches for testing purposes; however, a production ready product may have inputs coming directly from the medical monitors and thus replace the switches implemented for testing the prototype as shown in FIG. 13.

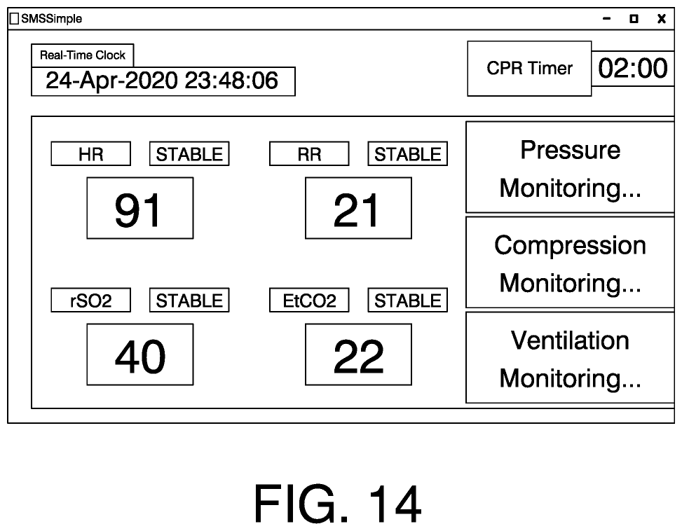

Referring to FIG. 14, a prototype GUI was developed to present preferred specifications as shown. CPR Quality was implemented by displaying the four major medical measurements:

Heart Rate, Respiratory Rate, rSO₂, and EtCO₂. These values are analyzed and the quality of CPR were displayed on the right side of the GUI in actions of “Pressure”, “Compression”, and “Ventilation.” “Pressure” is the place holder for alerts for increasing or decreasing the pressure of compressions on the patient's chest if any of the four major medical measurements need a pressure modification to stabilize. “Compression” is the place holder for alerts for increasing or decreasing the speed of compressions on the patient's chest if any of the four major medical measurements need a pressure modification to stabilize. “Ventilation” is the place holder for alerts for increasing the amount of oxygen flow to the patient. CPR Quality is 100% implemented in the “Smart Medical System.” Heart Rate is implemented by displaying its value directly on the GUI. The two notifications of “Pressure” and “Compression” produce the alerts needed to optimize CPR and stabilize the Heart Rate. This specification is also 100% implemented in the present invention in conjunction with the CPR Quality specification of the latter. Ventilation is implemented by displaying the Respiratory Rate value directly on the GUI. The notification of “Ventilation” produces the alert needed to optimize Ventilation and stabilize Ventilation. This specification is also 100% implemented in the present invention in conjunction with the CPR Quality specification.

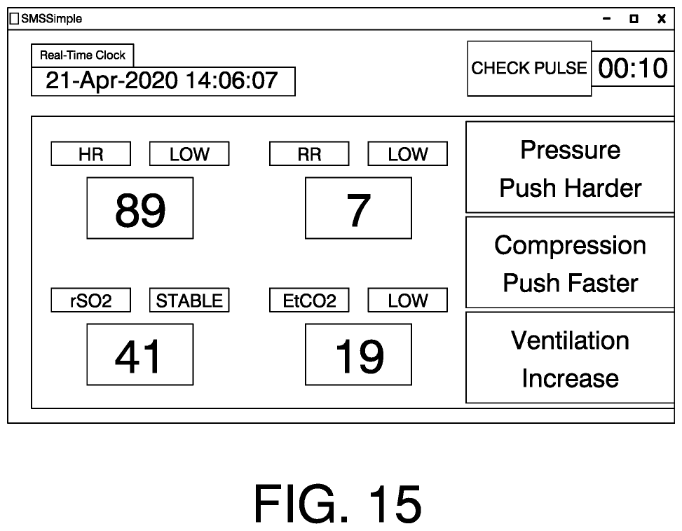

Referring now to FIG. 15, two additional preferred specifications are implemented in the prototype GUI of the present invention. The first of the two is Return of Spontaneous Circulation (ROSC). A ten second pulse check timer was implemented into the GUI, displayed at the top right of FIG. 15, to tell the responder all measurements are stabilized and the responder needs to check for a pulse for ten seconds. The handler exists in the Property Inspector of the timer. The handler of the timer was coded with a ten second pulse check timer in the functioning code. The second of the two is under the Other Recommendations. A two-minute CPR timer was implemented into the GUI, displayed at the top right of FIG. 14, to tell the responder performing CPR to switch with another responder so they do not become fatigued from applying compressions to the patient's chest. The handler exists in the Property Inspector of the timer. The handler of the timer would be almost identical with modification that the ten-minute timer would be for ten seconds and the two-minute timer would be for 120 seconds. The alert may be implemented as flashing buttons on the top right of the GUI. Other alerts types are possible.

Referring to FIG. 16, a prototype design of the present invention (10, FIG. 1; 20, FIG. 2) is shown where all five specifications previously mentioned have both visual and audio alert capability. The specifications can be viewed on the screen and they can be heard. The GUI organizes the information and displays the four major medical measurements of the patient for ease of viewing for the responder. The present invention (10, FIG. 1; 20, FIG. 2) combines AI enabled software and purpose built hardware, where the hardware implemented in the prototype design consists of a seven-inch display, a capacitive touch screen overlay to enable touch screen capabilities, and the LattePanda V1.0 with 4G/64 GB storage. The assembled prototype in FIG. 16 shows the screen powered on by the microprocessor. Not pictured is a 5 Volt/2 Amp adapter and USB end of an Arduino Mini wire to power the device from a 120 Volt outlet. To protect this entire assembly, an enclosure was designed to hold the screen and the microprocessor. The execution of the assembly in a finished product could be 3D printed or injection molded using high impact polymer materials typical for electronic medical devices and compliant with industry standards.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. An assistive device for guiding delivery and evaluating performance of cardiopulmonary resuscitation (CPR) during cardiac arrest (CA), comprising:
an electronic interface for receiving signal input from any of multiple invasive and noninvasive biometric monitoring devices that measure patient vital signs;
a processing device including machine learning algorithms that evaluate patient vital signs against personalized boundary parameter values compliant with American Heart Association (AHA) Pediatric Advanced Life Support (PALS) protocols;
a Graphical User Interface (GUI) that delivers in substantially realtime actionable visual and aural guidance and feedback including at least one of limiting or actuating changes and rate of changes of at least chest compression to affect alteration in patient vital signs, said guidance including corrective indicators directed to CPR performance adjustments to achieve therapeutic motion technique responsive to deviation from said personalized boundary values.

2. The assistive device of claim 1, wherein input from said multiple monitoring devices is combined and processed using artificial intelligence (AI) techniques to provide performance guidance displayed in said GUI on a single monitor during CPR.

3. The assistive device of claim 1, wherein inputs to said device include at least (a) heart rate, (b) end-tidal carbon dioxide—ETCO2, and (c) regional cerebral oxygen saturation—RSO2, which together are processed by said device to evaluate effectiveness of ongoing CPR and provide performance indicators of delivered CPR quality for a patient experiencing CA.

4. The assistive device of claim 1, further comprising an artificial intelligence function, that evaluates effectiveness of CPR in real time against AHA PALS protocols as CPR is performed and provides actionable guidance directed to increasing effectiveness of CPR being delivered.

5. The assistive device of claim 1, wherein said interface imports data as inputs from at least three types of monitoring devices, including any of Volumetric Capnography monitor, Near Infrared Spectroscopy monitor, and Arterial Line sensing monitor.

6. The assistive device of claim 1, wherein outputs are produced that include at least (a) performance parameters for compressions (e.g., press harder, softer, or keep doing what you're doing), (b) ventilation effectiveness, and (c) indication if return of spontaneous circulation (ROSC) has occurred.

7. The assistive device of claim 6, wherein said GUI is specifically configured to present said outputs.

8. The assistive device of claim 1, wherein said device is adapted for use in either a relatively fixed institutional setting including at least an emergency room (ER) and an ICU, or a dynamic environment including at least emergency vehicles and personal mobile devices.

9. An assistive device for guiding delivery and evaluating performance of cardiopulmonary resuscitation (CPR) during cardiac arrest (CA), comprising:
an electronic interface for receiving signal input from any of multiple invasive and noninvasive biometric monitoring devices that measure patient vital signs, said monitoring device types including any of Volumetric Capnography monitor, Near Infrared Spectroscopy monitor, and Arterial Line sensing monitor;
a processing device including machine learning algorithms that evaluate patient vital signs against personalized boundary parameter values compliant with American Heart Association (AHA) Pediatric Advanced Life Support (PALS) protocols, said boundary parameters including at least heart rate, end-tidal carbon dioxide—ETCO₂, and regional cerebral oxygen saturation—RSO₂;
a Graphical User Interface (GUI) that delivers in substantially realtime actionable visual and aural guidance and feedback including at least one of limiting or actuating changes and rate of changes of at least chest compression to affect alteration in patient vital signs, said guidance including corrective indicators directed to CPR performance adjustments to achieve therapeutic motion technique responsive to deviation from said personalized boundary values,
wherein said corrective indicators include at least performance parameters for compressions (e.g., press harder, softer, or keep doing what you're doing), and
wherein said quality of delivered CPR is measured by ventilation effectiveness, and return of spontaneous circulation (ROSC).

10. A method for guiding delivery and evaluating performance of cardiopulmonary resuscitation (CPR) during cardiac arrest (CA), comprising:
receiving signal input from any of multiple invasive and noninvasive biometric monitoring devices that measure patient vital signs, said monitoring device types including any of Volumetric Capnography monitor, Near Infrared Spectroscopy monitor, and Arterial Line sensing monitor;
evaluating received patient vital sign data against personalized boundary parameter values compliant with American Heart Association (AHA) Pediatric Advanced Life Support (PALS) protocols, said boundary parameters including at least heart rate, end-tidal carbon dioxide—ETCO₂, and regional cerebral oxygen saturation—RSO₂;
providing a Graphical User Interface (GUI) that delivers in substantially realtime actionable visual and aural guidance and feedback including at least one of limiting or actuating changes and rate of changes of at least chest compression to affect alteration in patient vital signs, said guidance including corrective indicators directed to CPR performance adjustments to achieve therapeutic motion technique responsive to deviation from said personalized boundary values,
wherein said corrective indicators include at least performance parameters for compressions (e.g., press harder, softer, or keep doing what you're doing), and
wherein said quality of delivered CPR is measured by ventilation effectiveness, and return of spontaneous circulation (ROSC).
